(19) United States
(12) Patent Application Publication
(10) Pub. No.: US 2021/0228121 A1
(43) Pub. Date: Jul. 29, 2021

(54) VASCULAR ACCESS DEVICE HAVING A VENTED BLOOD COLLECTION PORT

(71) Applicant: Becton, Dickinson and Company, Franklin Lakes, NJ (US)

(72) Inventors: Jonathan Karl Burkholz, Salt Lake City, UT (US); Weston F. Harding, Lehi, UT (US); Megan Scherich, Salt Lake City, UT (US)

(21) Appl. No.: 17/143,991

(22) Filed: Jan. 7, 2021

Related U.S. Application Data
(60) Provisional application No. 62/967,479, filed on Jan. 29, 2020.

(51) Int. Cl. A61B 5/15; A61M 39/10; A61M 25/00

(52) U.S. Cl. CPC A61B 5/150221; A61M 39/105; A61B 5/150992; A61M 2039/1072; A61B 5/150213; A61M 2039/1077; A61M 25/0023

(57) ABSTRACT

A vascular access device may have a vented blood collection port. The vented blood collection port can be provided on a side port of the vascular access device that is positioned between a catheter adapter and an extension set. The side port provides separate fluid pathways for collecting blood and injecting fluids to thereby isolate the blood from the extension set. The vented blood collection port may be provided in the form of a septum housing that moves from a blood collection position to a fluid injection position. When in the blood collection position, the septum housing may enable the extension set to be primed and remain primed during a blood draw.

### RELATED APPLICATIONS

This application claims the benefit of U.S. Provisional Patent Application No. 62/967,479, filed Jan. 29, 2020, and entitled VASCULAR ACCESS DEVICE HAVING A VENTED BLOOD COLLECTION PORT, which is incorporated herein in its entirety.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is an over-the-needle peripheral intravenous (“IV”) catheter (“PIVC”). As its name implies, the over-the-needle catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into the vasculature of the patient.

An integrated PIVC is a PIVC having an integrated extension set. Such extension sets typically consist of extension tubing that is integrated at one end into the catheter adapter and that includes an access port (e.g., a luer connector) coupled to the other end. Integrated PIVCs are oftentimes used to draw blood. For example, after inserting the catheter of the integrated PIVC into the patient's vasculature, a clinician may allow blood to flow into the extension set up to the access port. To enable this blood flow, a vent plug will typically be coupled to the access port, which will allow air to escape the extension tube as the blood flows into the extension tube. Once the blood has flowed up to the access port, the clinician will then remove the vent plug and attach a blood collection set (e.g., a vacuum tube adapter) in its place. The blood can then be collected.

The process of collecting blood through the extension set of an integrated PIVC has various drawbacks. For example, before blood can be collected, the entire extension set must be primed (i.e., air must be vented from the access port to allow blood to flow into the extension set up to the access port). Additionally, the removal of the vent plug from the access port exposes the fluid pathway to the external environment. The subsequent attachment of the blood collection set could therefore contaminate the fluid pathway. As a result, the clinician may need to sterilize the access port before attaching the blood collection set thereby prolonging the blood collection process. Due to the length of the extension tubing, it can take a substantial amount of time for the blood to flow into the blood collection set, particularly when the patient's blood pressure is low. Once blood is collected, there will be residual blood within the extension set. Although fluid could be injected through the access port to flush the blood from the extension tubing, it is difficult to fully flush residual blood that may be trapped within the access port. This residual blood could increase the risk of bloodstream infection (BSI) since the same access port is also typically used to inject fluids into the patient's vasculature.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to a vascular access device having a vented blood collection port. The vented blood collection port can be provided on a side port of the vascular access device that is positioned between a catheter adapter and an extension set. The side port provides separate fluid pathways for collecting blood and injecting fluids to thereby isolate the blood from the extension set. The vented blood collection port may be provided in the form of a septum housing that moves from a blood collection position to a fluid injection position. When in the blood collection position, the septum housing may enable the extension set to be primed and remain primed during a blood draw.

In a first set of example embodiments, a vascular access device may include a catheter assembly from which a catheter extends distally, a side port having a distal end that couples to the catheter assembly via a side inlet of the catheter assembly and a septum housing. The side port also includes a side branch to which an extension set is coupled. The septum housing is coupled to the proximal end of the side port and includes a septum. The septum housing is configured to move from a blood collection position to a fluid injection position. When the septum housing is in the blood collection position, the septum housing blocks a fluid pathway between the distal end of the side port and the side branch. When the septum housing is in the fluid injection position, the septum housing does not block the fluid pathway between the distal end of the side port and the side branch.

In the first set of example embodiments, the septum housing may be configured to rotate from the blood collection position to the fluid injection position. In such embodiments, when the septum housing is in the blood collection position, the septum may be configured to cover an opening of the side branch, whereas, when the septum housing is in the fluid injection position, the septum may be configured to not cover the opening of the side branch. As an example, the septum may have an angled distal end. The vascular access device may also have a first channel that vents air from within the side port when the septum housing is in the blood collection position, and a second channel that vents air from within the extension set when the septum housing is the blood collection position. The first channel may be formed in a sidewall of the side port and the second channel may be formed in the septum.

In the first set of example embodiments, the septum housing may be configured to move linearly from the blood collection position to the fluid collection position. In such embodiments, the septum housing may include a septum housing sidewall having an opening. When the septum housing is in the blood collection position, the opening in the septum housing sidewall may be configured to not overlap with an opening of the side branch, whereas, when the septum housing is in the fluid injection position, the opening in the septum housing sidewall may be configured to overlap with the opening in the side branch. In such embodiments, the vascular access device may include a first channel that vents air from within the side port when the septum housing is in the blood collection position, and a second channel that vents air from within the extension set when the septum housing is the blood collection position. The first channel may be formed in the septum housing sidewall and the second channel may be formed in a sidewall of the side port. The septum housing may include one or more seals that block the first and second channels when the septum housing is in the fluid injection position.

In a second set of example embodiments, a vascular access device may include a catheter assembly from which a catheter extends distally, a side port having a distal end that is coupled to the catheter assembly via a side inlet of the catheter assembly and a vented blood collection port formed at the proximal end of the side port. The side port can also include a side branch to which an extension set is coupled.

In the second set of example embodiments, the vented blood collection port may be in the form of a septum housing that is configured to move from a blood collection position to a fluid injection position. When the septum housing is in the blood collection position, the septum housing blocks a fluid pathway between the distal end of the side port and the side branch. When the septum housing is in the fluid injection position, the septum housing does not block the fluid pathway between the distal end of the side port and the side branch.

In the second set of example embodiments, the vented blood collection port may be in the form of a removable vent plug having a blood collection channel and a priming fluid venting channel. The blood collection channel vents air from within the side port and the priming fluid venting channel vents air from within the extension set.

In the second set of example embodiments, the vascular access device may further include a valve positioned in the side branch. The valve can be configured to selectively block a fluid pathway between the distal end of the side port and the side branch.

In a third set of example embodiments, a vascular access device may include a catheter adapter and a side port having a distal end coupled to the catheter adapter, a proximal end forming a vented blood collection port and a side branch to which an extension set is connected. The vented blood collection port may be in the form of a septum housing that is configured to move from a blood collection position to a fluid injection position. In such cases, when the septum housing is in the blood collection position, the septum housing blocks a fluid pathway between the distal end of the side port and the side branch, whereas, when the septum housing is in the fluid injection position, the septum housing does not block the fluid pathway between the distal end of the side port and the side branch. The septum housing may include a septum. In such cases, when the septum housing is in the blood collection position, the septum may cover an opening of the side branch, whereas, when the septum housing is in the fluid injection position, the septum may not cover the opening of the side branch.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present disclosure. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 illustrates a peripheral intravenous catheter having a vented blood collection port in accordance with embodiments of the present disclosure;

FIGS. 1A and 1B each provide a cross-sectional view of the vented blood collection port of FIG. 1;

FIGS. 2A-2E illustrate a sequence for collecting a blood sample using the peripheral intravenous catheter of FIG. 1;

FIGS. 3A and 3B provide cross-sectional views of a vented blood collection port of another vascular access device that is configured in accordance with embodiments of the present disclosure;

FIGS. 4A and 4B provide cross-sectional views of a vented blood collection port of another vascular access device that is configured in accordance with embodiments of the present disclosure;

FIGS. 5A and 5B provide cross-sectional views of a vented blood collection port of another vascular access device that is configured in accordance with embodiments of the present disclosure;

FIG. 6 provides a cross-sectional view of a vented blood collection port of another vascular access device that is configured in accordance with embodiments of the present disclosure; and

FIG. 7 illustrates a vented blood collection port of another vascular access device that is configured in accordance with embodiments of the present disclosure.

### DESCRIPTION OF EMBODIMENTS

Embodiments of the present disclosure will primarily be described in the context of integrated PIVCs. However, embodiments of the present disclosure equally extend to other integrated vascular access devices. For purposes of the specification and the claims, an integrated vascular access device should be construed as a vascular access device that includes an integrated extension set.

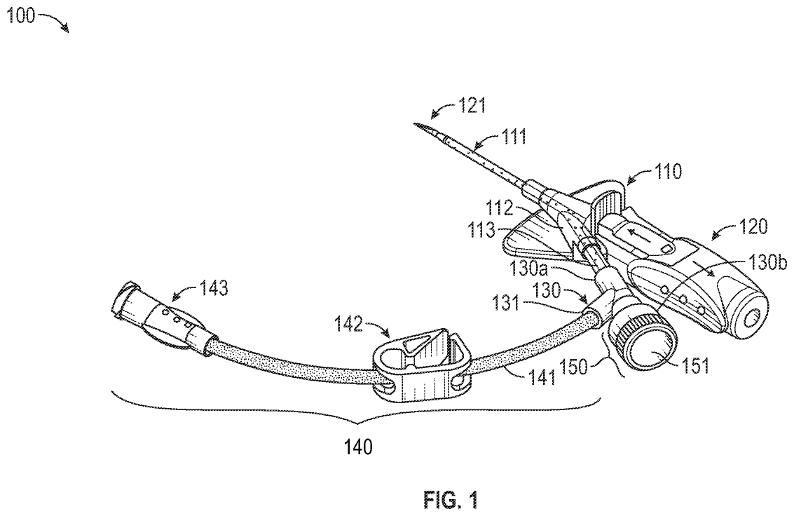

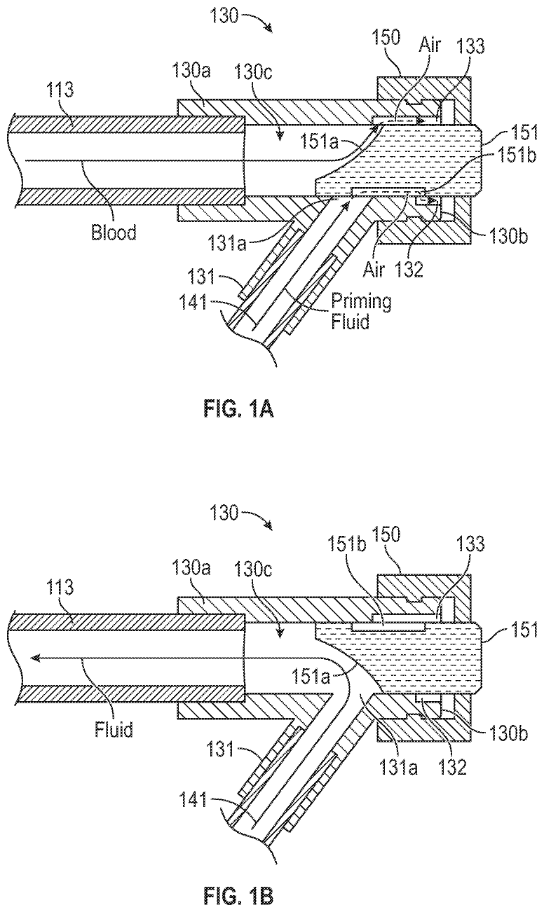

FIG. 1 provides an example of a PIVC 100 that is configured in accordance with some embodiments of the present disclosure. PIVC 100 includes a catheter adapter 110 from which a catheter 111 distally extends and a needle assembly 120 from which a needle 121 distally extends. Prior to insertion, needle assembly 120 is coupled to catheter adapter 110 to cause needle 121 to extend distally from catheter 111. Catheter adapter 110 also includes a side inlet 112 which defines a fluid pathway into and out from catheter 111. The exact configuration and function of catheter adapter 110 and needle assembly 120 are not essential to the present disclosure and any suitable configuration and/or interaction of these components could be employed.

PIVC 100 also includes a side port 130 having a distal end 130a that is coupled to side inlet 112 via intermediate tubing 113, a proximal end 130b to which a septum housing 150 is coupled and a side branch 131 by which an extension set 140 is integrated with side port 130. As depicted, extension set 140 can include extension tubing 141 that extends between side branch 131 and an access port 143 and a pinch clamp 142 for occluding extension tubing 141. It is noted, however, that many different types and configurations of extension sets could be used with embodiments of the present disclosure. Of importance is that side port 130 is positioned between extension set 140 and catheter adapter 110.

Side port 130 defines two fluid pathways. One fluid pathway extends between distal end 130a and proximal end 130b. As described in detail below, this fluid pathway may be used to collect a blood sample through PIVC 100 by connecting a blood collection set/adapter to proximal end 130b (e.g., via septum housing 150). The other fluid pathway extends between distal end 130a and side branch 131 and is therefore the fluid pathway into and out from extension set 140.

In the depicted example, side branch 131 has a proximally-directed orientation and is therefore oriented towards septum 151 of septum housing 151. This proximally-directed orientation causes fluid flowing from extension set 140 to be directed against septum 151 to thereby flush residual blood or other fluid from septum 151. However, side branch 131 may equally have a distally-directed orientation or a perpendicular orientation.

FIGS. 1A and 1B provide cross-sectional views of side port 130 when septum housing 150 is in a blood collection position and a fluid injection position respectively. As shown, septum housing 150 contains a septum 151 having an angled distal end 151a. Septum housing 150 is coupled to proximal end 130b of side port 130 in a manner that allows septum housing 150 to be rotated relative to side port 130. Additionally, septum 151 can be positioned within septum housing 150 such that septum 151 will rotate as septum housing 150 is rotated.

The blood collection position of septum housing 150 depicted in FIG. 1A may typically represent the initial position of septum housing 150 (e.g., the position of septum housing 150 when needle 121 and catheter 111 are inserted into the patient's vasculature). In this blood collection position, angled distal end 151a is oriented away from side branch 131 such that septum 151, or more specifically, the longer portion of septum 151, blocks the fluid pathway between the interior 130c of side port 130 and extension tubing 141. As labeled, this will allow extension set 140 to be pre-primed with priming fluid. To facilitate this pre-priming, septum 151 can include a channel 151b that extends between the opening of side branch 131 and a channel 132 formed towards proximal end 130b of side port 130.

In some embodiments, channel 151b can be configured to allow air to vent from extension set 140 while blocking the passage of priming fluid. For example, channel 151b could be configured to overlap opening 131a of side branch 131 an amount that is sufficient only to vent air but not fluid. Alternatively or additionally, channel 151b could be filled with or formed of an air permeable material that blocks the passage of fluid. In other embodiments, channel 151b may not block the passage of fluid, while channel 132 could be configured to block the passage of fluid. For example, channel 132 could be filled with an air permeable material that blocks the passage of fluid. In any case, when septum housing 150 is in the blood collection position, air will be vented from extension set 140 to thereby allow extension set to be primed while blocking the priming fluid from flowing into interior 130c of side port 130.

When septum housing 150 is in this blood collection position, the orientation of angled distal end 151a of septum 151 will also cause a channel 133 formed in side port 130 to be exposed to interior 130c of side port 130 (i.e., angled distal end 151a causes septum 151 to not block channel 133). As labeled, this will allow air that is within interior 130c to be vented as blood flows from the patient's vasculature towards and into interior 130c. In some embodiments, channel 133 can be filled with an air permeable material that blocks the passage of blood to thereby prevent the blood from escaping through channel 133. Accordingly, with septum housing 150 in the blood collection position, proximal end 130b of side port 130 and septum housing 151 form a vented blood collection port. For example, a needle of a blood collection set could be inserted through septum 151 to collect a blood sample. Notably, due to angled distal end 151a of septum 151, this blood draw can be performed while extension set 140 is primed without the risk of diluting the blood sample with priming fluid.

Referring to FIG. 1B, septum housing 150 has now been rotated into the fluid injection position. As an example, a clinician could rotate septum housing 150 into this position after collecting a blood sample. As shown, in the fluid injection position, angled distal end 151a of septum 151 will now face towards side branch 131 such that septum 151 no longer blocks opening 131a of side branch 131. The fluid pathway between extension set 140 and side port 130 will therefore be opened. With the rotation of septum 151, channel 151b will no longer extend between the opening of side branch 131 and channel 132. Similarly, septum 151 will now block channel 133. Also, due to the orientations of side branch 131 and angled distal end 151a, fluid injected from extension set 151 will be directed against septum 151 thereby increasing the likelihood that any residual blood will be flushed from septum 151 and the surrounding surfaces.

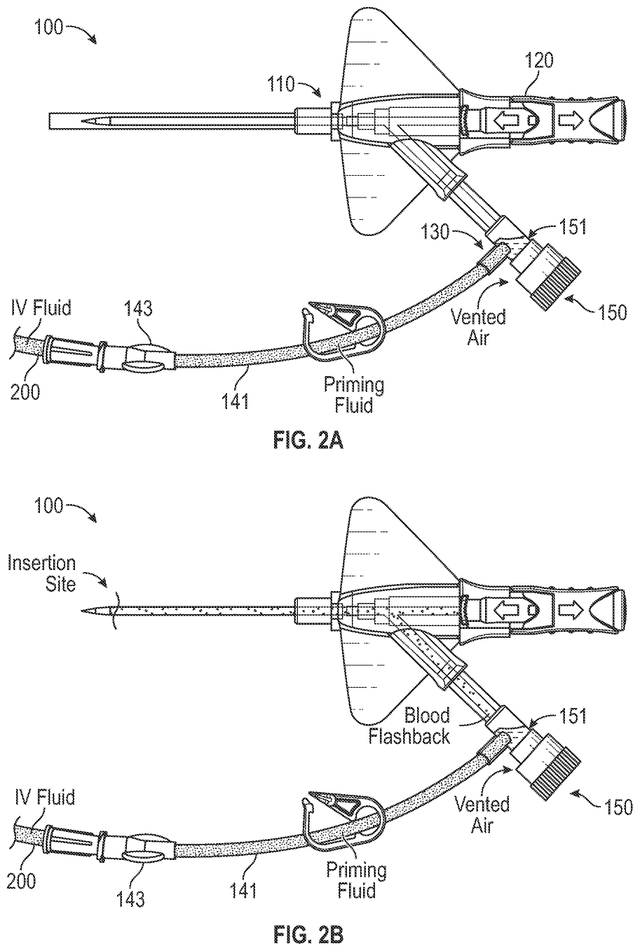

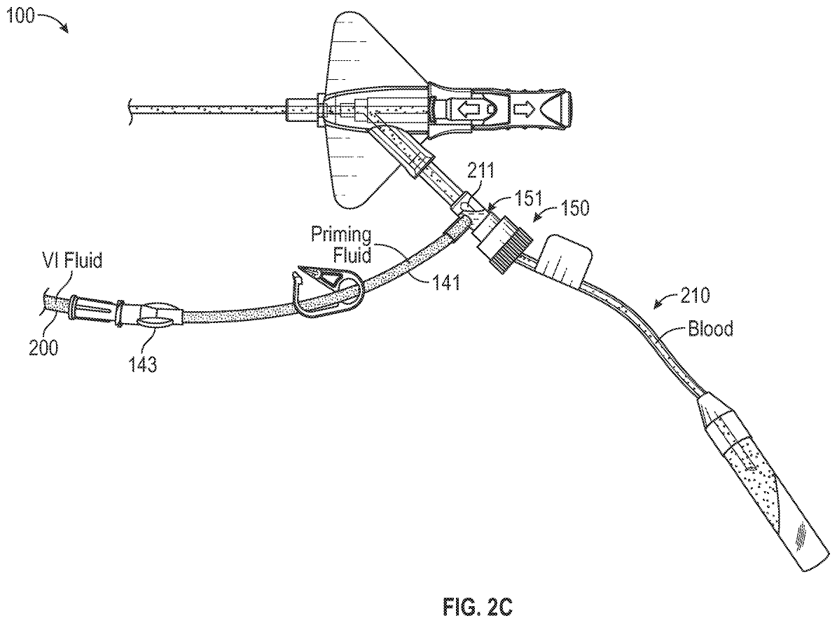

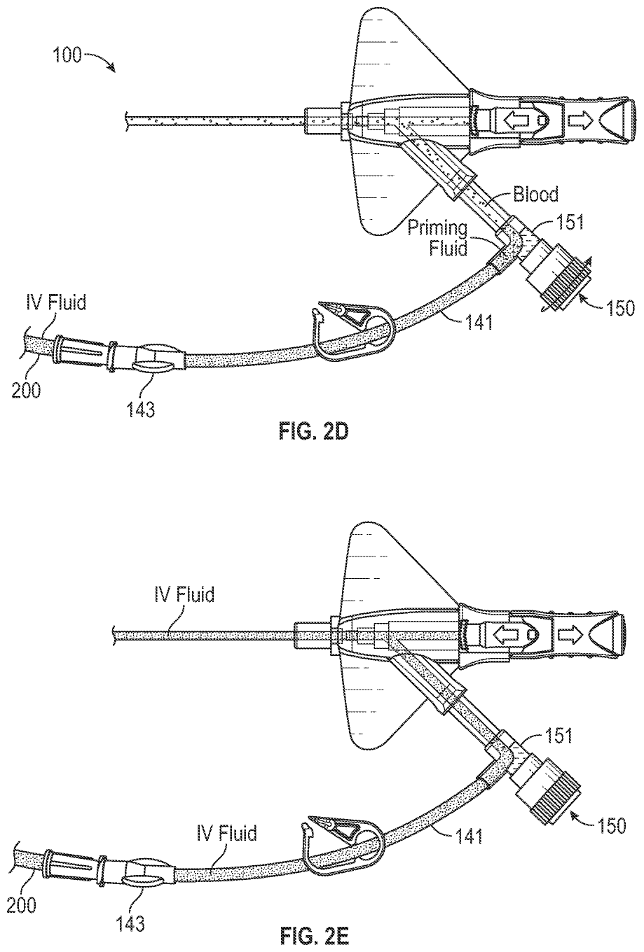

FIGS. 2A-2E illustrate a sequence of steps for collecting a blood sample using PIVC 100, according to some embodiments of the present disclosure. In FIG. 2A, PIVC 100 has not yet been inserted into the patient's vasculature but extension set 140 has been pre-primed with priming fluid. As represented, this pre-priming could be accomplished by connecting an IV set 200 to access port 143 but extension set 140 could be primed in any suitable manner. As described above, by positioning septum housing 150 in the blood collection position, air will be vented from extension set 140 as the priming/IV fluid fills extension set 140.

Referring to FIG. 2B, it is assumed that PIVC 100 has now been inserted into the patient's vasculature while extension set 140 is pre-primed. It is noted, however, that PIVC 100 could be inserted into the patient's vasculature before priming extension set 140. In any case, after inserting PIVC 100 into the patient's vasculature, and due to the venting of air provided through channel 133, blood flashback will flow towards and into side port 130.

Referring to FIG. 2C, typically after blood has filled side port 130, a clinician can connect a blood collection set 210 to side port 130. For example a needle 211 of blood collection set 210 can be inserted through septum 151 while septum housing 150 remains in the blood collection position. Blood collection set 210, which may include a vacuum tube, can then be used to collect the blood sample(s). Needle assembly 120 is shown as not yet having been removed from catheter adapter 110 to thereby represent that the blood collection could be performed before needle 121 is withdrawn from the patient's vasculature. However, the blood collection could equally be performed after removing needle assembly 120.

Referring to FIG. 2D, after the blood sample(s) have been collected, blood collection set 210 can be removed from side port 130. With extension set 140 already primed, the clinician can rotate septum housing 150 to the fluid injection position to thereby cause septum 151 to no longer block the fluid pathway out from extension set 140. As FIGS. 2D and 2E represent, this will cause the priming fluid/IV fluid to flow from extension set 140, through side port 130 and catheter adapter 110 and into the patient's vasculature. Notably, the flow of the priming fluid/IV fluid will flush the blood contained in side port 130, and particularly residual blood on septum 151, into the patient's vasculature.

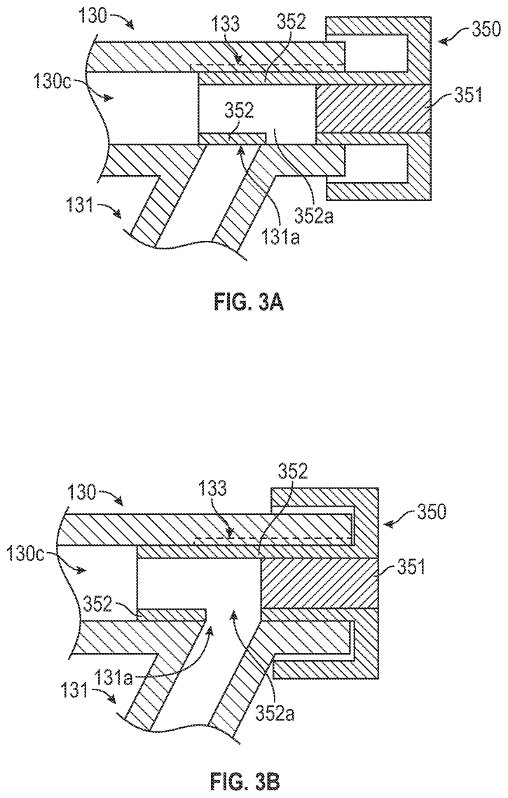

In the above-described embodiments, septum housing 150 has been configured to selectively block the fluid pathway from extension set 140 while selectively venting air from side port 130 using a rotation-based configuration. In contrast, in other embodiments, a linear-based configuration could be employed. For example, FIGS. 3A and 3B provide cross-sectional views of side port 130 with a septum housing 350. In contrast to septum housing 150, septum housing 350 is configured to slide between the blood collection position and the fluid injection position. In particular, septum housing 350 includes a septum 351 that is positioned towards and contained within a septum housing sidewall 352 that extends into side port 130.

FIG. 3A illustrates septum housing 350 in the blood collection position. As shown, septum housing sidewall 352 is inserted sufficiently into side port 130 to cover opening 131a of side branch 131 thereby blocking the fluid pathway out from extension set 140. In such embodiments, channel 133 can be configured to extend distally beyond septum housing sidewall 352 so that air can be vented out from interior 131 of side port 130. An opening 352a is formed in septum housing sidewall 352 but is positioned proximal to opening 131a of side branch 131 when septum housing 350 is in the blood collection position. Although not shown, side port 130 can include a channel (e.g., similar to channel 132) that vents air from extension set 140 when septum housing 350 is in the blood collection position.

With septum housing 350 in this blood collection position, a blood sample can be collected using a blood collection set that is inserted through septum 351. After collecting the blood sample and removing the blood collection set, a clinician can apply a distal force to septum housing 350 to cause it to slide into the fluid injection position shown in FIG. 3B. In this fluid collection position, opening 352a in septum housing sidewall 352 will align with (or at least overlap) opening 131a of side branch 131 thereby opening the fluid pathway from extension set 140. Sidewall 352 will also block channel 133. The priming fluid that may have been contained within extension set 140 and any fluid from a fluid source (e.g., an IV set) connected to access port 143 will then commence flowing from extension set 140 into the patient's vasculature thereby flushing the residual blood into the patient's vasculature.

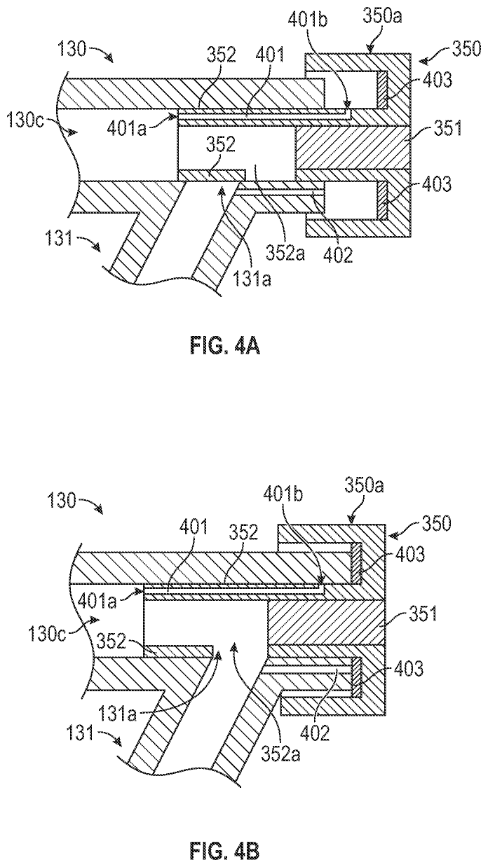

FIGS. 4A and 4B provide a variation of the embodiments represented in FIGS. 3A and 3B. In FIGS. 4A and 4B, septum housing 350 has the same general shape and functions in a similar manner to selectively open the fluid pathway from extension set 140. However, the embodiments represented in FIGS. 4A and 4B provide different means for venting air. In such embodiments, a channel 401 is formed within septum housing sidewall 352 and extends from a distal end 401a at the distal end of septum housing sidewall 352 to a proximal end 401b that is open to the external environment. For example, in FIG. 4A, proximal end 401b of channel 401 opens under cap portion 350a of septum housing 350 that surrounds proximal end 130b of side port 130. Air from within interior 130c will therefore vent out from cap portion 350a as blood flows towards septum 351. A channel 402 is also formed in a portion of side port 130 that extends between opening 131a of side branch 131 and proximal end 130b of side port 130. Channel 402 will therefore vent air from extension set 140 as extension set is primed. Either or both channels 401 and 402 may be filled with an air permeable material that blocks fluids in some embodiments.

The depicted positions and orientations of channels 401 and 402 represent just one suitable configuration of the channels. For example, in other embodiments, channel 401 could instead be formed within the sidewall of side port 130. In any case, to seal off channels 401 and 402 when septum housing 350 is moved into the fluid injection position, one or more sealing members 403 can be formed on or connected to the underside of cap portion 350a of septum housing 350. Sealing member(s) 403 can contact proximal end 130b of side port 130 to form an air tight seal that will prevent fluid and air from flowing out from proximal end 130b. Although not shown, side port 130 and/or septum housing 350 may include physical structures (e.g., threads) that tightly retain septum housing 350 in the blood collection position.

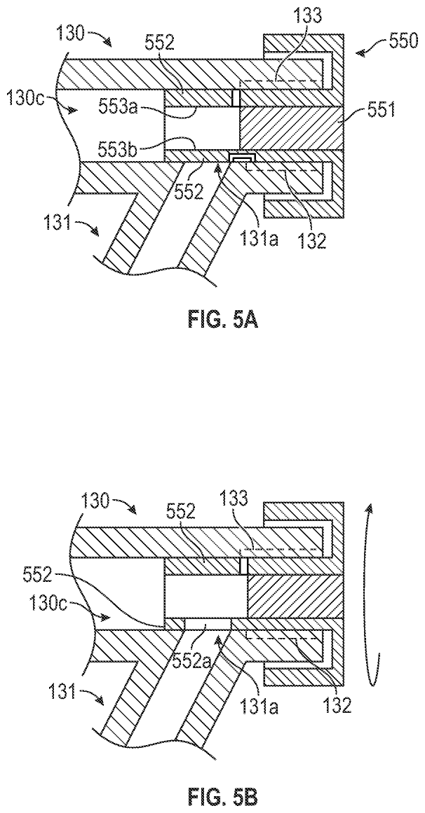

FIGS. 5A and 5B represent embodiments where side port 130 includes a septum housing 550 that rotates between the blood collection and fluid injection positions. Septum housing 550 is similar to septum housing 350 in that it includes a septum 551 that is positioned in a septum housing sidewall 552 having an opening 552a. FIG. 5A illustrates that, when septum housing 550 is in the blood collection position, opening 552a is not positioned overtop opening 131a of side branch 131. However, channels 553a and 553b that are formed within septum housing sidewall 552 are aligned with channels 132 and 133 respectively. Channel 553a extends from the inside to the outside of septum channel sidewall 552 and is positioned proximal to septum 551 to thereby vent air from within side port 130 into channel 133. Both ends of channel 553b extend through the outside of septum housing sidewall 552 with the distal end being positioned within opening 131a of side branch 131 and the proximal end aligning with channel 132 to thereby vent air from within extension set 140.

After collecting a blood sample with septum housing 550 in the blood collection position, the clinician can rotate septum housing 550 into the fluid injection position shown in FIG. 5B. In this fluid injection position, opening 552a will align with opening 131a of side branch 131 thereby opening the fluid pathway from extension set 140. Similarly, channels 553a and 553b will no longer align with channels 132 and 133.

In any of the above-described embodiments, side port 130 and/or the septum housing can be configured to provide a visual indication of whether the septum housing is in the blood collection position or the fluid injection position. Also, in any of the above-described embodiments, the septum housing can be configured to move repeatedly between the blood collection position and the fluid injection position or may be locked into the fluid injection position to prevent returning to the blood collection position.

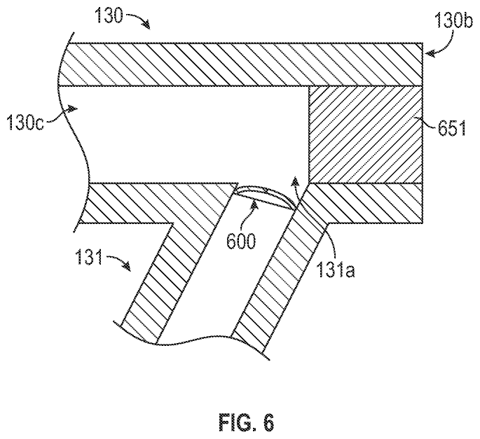

FIG. 6 illustrates an example where side port 130 does not employ a septum housing. Instead, septum 651 is contained directly within proximal end 130b of side port 130. Also, a one-way valve 600 is positioned within opening 131a of side branch 131. Although not shown, side port 130 could include channels for venting air from interior 130c of side port 130 and/or side branch 131. One-way valve 600 may be in the form of a domed-elastomeric member where the concave side of the domed-elastomeric member faces extension set 140. With this configuration, one-way valve 600 can flex open in response to an increase in fluid pressure that results when an IV set or other fluid source is delivering fluid through access port 143. In contrast, when blood flows into side port 130, any increase in fluid pressure will not be sufficient to bypass one-way valve 600. In other embodiments, other types of valves could be employed within opening 131a of side branch 131 to block the flow of priming fluid until after a blood sample has been drawn. For example, a door valve or a sliding valve could be used in place of one-way valve 600.

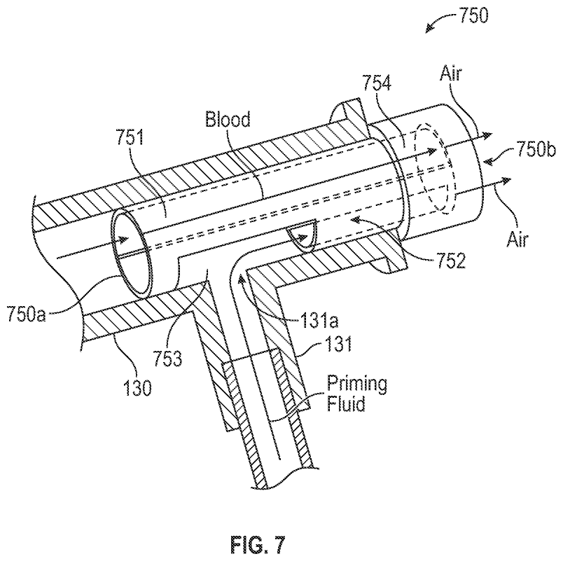

FIG. 7 provides an example where a removable vent plug 750 is used in place of a septum housing to provide a vented blood collection port on side port 130. Removable vent plug 750 is split into two separate lengthwise channels. A blood collection channel 751 extends between a distal end 750a and a proximal end 750b of removable vent plug 750. Blood collection channel 751 may include an air permeable material 754 that allows air to vent out proximal end 750b and which allows a blood collection set to be inserted into blood collection channel 751. In some embodiments, a septum could be positioned within blood collection channel 751. A priming fluid vent channel 752 is positioned opposite blood collection channel 751 and extends between a side opening 753 in removable vent plug 750 and proximal end 750b. Side opening 753 does not extend to distal end 750a such that priming fluid vent channel 752 is isolated from the blood flashback. Priming fluid vent channel 752 can contain an air permeable material that vents air but blocks fluid to thereby facilitate priming of extension set 140.

In FIG. 7, removable vent plug 750 is shown in the blood collection position. After a blood sample is collected through blood collection channel 751, the clinician could pull removable vent plug 750 in a proximal direction to remove it from side port 130 and then attach a needleless connector or other adapter.

To summarize, embodiments of the present disclosure enable a blood sample to be collected at the time of catheter placement via a vented blood collection port that is positioned distal to the access port (e.g., a needleless connector) of an extension set. With such designs, blood is isolated from the access port of the extension set. Such embodiments also enhance blood flashback and removable of residual blood after the blood collection process. In addition to enabling a blood sample to be collected at the time of catheter placement, embodiments of the present disclosure also enable a blood sample to be collected throughout the dwell time of the catheter by merely returning the septum housing to the blood collection position.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

1. A vascular access device comprising:
a catheter assembly from which a catheter extends distally, the catheter assembly having a side inlet;
a side port having a distal end that is coupled to the catheter assembly via the side inlet, a proximal end and a side branch to which an extension set is coupled; and
a septum housing that is coupled to the proximal end of the side port, the septum housing having a septum and being configured to move from a blood collection position to a fluid injection position, wherein, when the septum housing is in the blood collection position, the septum housing blocks a fluid pathway between the distal end of the side port and the side branch, and wherein, when the septum housing is in the fluid injection position, the septum housing does not block the fluid pathway between the distal end of the side port and the side branch.

2. The vascular access device of claim 1, wherein the septum housing is rotated from the blood collection position to the fluid injection position.

3. The vascular access device of claim 2, wherein, when the septum housing is in the blood collection position, the septum covers an opening of the side branch, and wherein, when the septum housing is in the fluid injection position, the septum does not cover the opening of the side branch.

4. The vascular access device of claim 3, wherein the septum has an angled distal end.

5. The vascular access device of claim 4, further comprising:
a first channel that vents air from within the side port when the septum housing is in the blood collection position; and
a second channel that vents air from within the extension set when the septum housing is the blood collection position.

6. The vascular access device of claim 5, wherein the first channel is formed in a sidewall of the side port and the second channel is formed in the septum.

7. The vascular access device of 2, wherein the septum housing includes a septum housing sidewall having an opening, and wherein, when the septum housing is in the blood collection position, the opening in the septum housing sidewall does not overlap with an opening of the side branch, whereas, when the septum housing is in the fluid injection position, the opening in the septum housing sidewall overlaps with the opening in the side branch.

8. The vascular access device of claim 7, further comprising:
a first channel formed in the septum hosing sidewall that vents air from within the side port when the septum housing is in the blood collection position; and
a second channel formed in the septum housing sidewall that vents air from within the extension set when the septum housing is the blood collection position.

9. The vascular access device of claim 1, wherein the septum housing is moved linearly from the blood collection position to the fluid collection position.

10. The vascular access device of claim 9, wherein the septum housing includes a septum housing sidewall having an opening, and wherein, when the septum housing is in the blood collection position, the opening in the septum housing sidewall does not overlap with an opening of the side branch, whereas, when the septum housing is in the fluid injection position, the opening in the septum housing sidewall overlaps with the opening in the side branch.

11. The vascular access device of claim 10, further comprising:
a first channel that vents air from within the side port when the septum housing is in the blood collection position; and
a second channel that vents air from within the extension set when the septum housing is the blood collection position.

12. The vascular access device of claim 11, wherein the first channel is formed in the septum housing sidewall and the second channel is formed in a sidewall of the side port.

13. The vascular access device of claim 12, wherein the septum housing includes one or more seals that block the first and second channels when the septum housing is in the fluid injection position.

14. A vascular access device comprising:
a catheter assembly from which a catheter extends distally, the catheter assembly having a side inlet;
a side port having a distal end that is coupled to the catheter assembly via the side inlet, a proximal end and a side branch to which an extension set is coupled; and
a vented blood collection port formed at the proximal end of the side port.

15. The vascular access device of claim 14, wherein the vented blood collection port comprises a septum housing that is configured to move from a blood collection position to a fluid injection position, wherein, when the septum housing is in the blood collection position, the septum housing blocks a fluid pathway between the distal end of the side port and the side branch, and wherein, when the septum housing is in the fluid injection position, the septum housing does not block the fluid pathway between the distal end of the side port and the side branch.

16. The vascular access device of claim 14, wherein the vented blood collection port comprises a removable vent plug having a blood collection channel and a priming fluid venting channel, the blood collection channel venting air from within the side port and the priming fluid venting channel venting air from within the extension set.

17. The vascular access device of claim 14, further comprising:
a valve positioned in the side branch, the valve selectively blocking a fluid pathway between the distal end of the side port and the side branch.

18. A vascular access device comprising:
a catheter adapter; and
a side port having a distal end coupled to the catheter adapter, a proximal end forming a vented blood collection port and a side branch to which an extension set is connected.

19. The vascular access device of claim 18, wherein the vented blood collection port comprises a septum housing that is configured to move from a blood collection position to a fluid injection position, wherein, when the septum housing is in the blood collection position, the septum housing blocks a fluid pathway between the distal end of the side port and the side branch, and wherein, when the septum housing is in the fluid injection position, the septum housing does not block the fluid pathway between the distal end of the side port and the side branch.

20. The vascular access device of claim 19, wherein the septum housing includes a septum, and wherein, when the septum housing is in the blood collection position, the septum covers an opening of the side branch, and wherein, when the septum housing is in the fluid injection position, the septum does not cover the opening of the side branch.
